(19) United States
(12) Patent Application Publication
(10) Pub. No.: US 2025/0275846 A1
(43) Pub. Date: Sep. 4, 2025

(54) EMBOLIC PROTECTION FOR MITRAL AND TRICUSPID VALVE PROCEDURES

(71) Applicant: Emboline, Inc., Santa Cruz, CA (US)

(72) Inventor: Amir BELSON, Savyon (IL)

(21) Appl. No.: 18/857,805

(22) PCT Filed: Apr. 13, 2023
(86) PCT No.: PCT/US2023/018438

§ 371 Date: Oct. 17, 2024

Related U.S. Application Data
(60) Provisional application No. 63/331,836, filed on Apr. 17, 2022.

(51) Int. Cl. A61F 2/01

(52) U.S. Cl. CPC A61F 2/013; A61F 2230/008; A61F 2250/0059

(57) ABSTRACT

Emboli released while treating an atrial heart valve between a patient's atrium and ventricle may be captured by placing a filter in or over a ventricular heart valve which establishes blood flow from the patient's ventricle into the patient's vasculature. The atrial heart valve my be treated while the filter remains over the ventricular heart valve. Emboli released into the ventricle while treating the atrial heart valve are captured by the filter during ventricular systole when blood flows from the ventricle through the filter into the vasculature. The filter may be removed together with the captured emboli after the treatment is completed.

### CROSS-REFERENCE

This application claims the benefit of U.S. Provisional No. 63/331,836 (Attorney Docket No. 41959-716.101), filed Apr. 17, 2022, the entire content of which is incorporated herein by reference.

### BACKGROUND OF THE INVENTION

1. Field of the Invention. The present invention relates generally to medical methods and apparatus and, more particularly, to methods and devices for inhibiting the release of emboli into a patient's vasculature during cardiac valve implantation and repair procedures.

The mitral valve and the tricuspid valve control blood flow from a patient's left and right atriums to the patient's left and right ventricles, respectively. Beating heart procedures on these valves, referred to hereinafter as the atrial outlet valves, can cause the release of debris and emboli into the ventricles and then into blood circulation through the aortic and pulmonary valves, respectively, referred to hereinafter as the ventricular outlet valves. Such release of emboli through the aortic and pulmonary valves potentially occludes the distal blood vessels which can cause tissue damage.

Current aortic valve emboli protection protocols often rely on arterial access to introduce a filter assembly into the patient's aorta, downstream of the aortic valve. Protection of a patient's pulmonary vasculature is even more problematic as there is no easy access to the pulmonary arteries which are down stream of the pulmonary valve.

For these reasons, it would be desirable to provide methods and apparatus for capturing emboli released during cardiac valve procedures performed on beating heart, such as closed chest, procedures performed on a patient's mitral valve or tricuspid valve. The methods and apparatus for capturing emboli should be compatible with a wide variety of cardiac procedures, including but not limited to prosthetic valve implantation, valve repair, and the like. It would be further desirable if methods and apparatus for capturing emboli in a beating heart could be incorporated into apparatus intended for use with separate cardiac implantation and repair apparatus as well as being suitable for incorporation into novel cardiac implantation and repair apparatus. At least some of these objectives will be met by the methods and apparatus of the present invention as described and claimed herein.

### SUMMARY OF THE INVENTION

The present invention provides methods and apparatus for embolic protection during cardiac procedures being performed on atrial outlet valves, i.e., mitral valves and tricuspid valves. Filters, such as basket-shaped filters, having an open end configured to face “downstream” so that blood flows through the filter are placed over or in an associated ventricular outlet valve, i.e., mitral valves and aortic valves, respectively. In this way, emboli released by the procedure may be carried into a ventricle but will be captured in the filter before entering a patient's vasculature or lungs.

In some embodiments, the opening will have a circumference equal to or larger than an inner circumference a of a target vessel (aorta or pulmonary artery).

In some embodiments, the opening will have a circumference sufficient large to cover and preferably extend beyond the circumference of the opening of a target vessel (aorta or pulmonary artery) in the heart chamber wall, usually being sufficiently large to cover an annular margin surrounding the valve opening of at least 1 mm, 2 mm, 3 mm, 4 mm, 5 mm, 1 cm or greater, usually being in a range from 1 mm to 1 cm, more usually from 2 mm to 1 cm, often from 3 mm to 8 mm.

The basket will comprise a foraminous filter body or wall having pores large enough to allow blood flow while being sufficiently small to block at least most dangerous emboli, typically being in a range from 100 μm to 200 μm (microns).

Optionally, the filter may be coated with an anti-platelet aggregation agent, such as heparin.

The basket will often be configured to be expanded by a balloon, typically being formed form a biocompatible, malleable metal (e.g. a stainless-steel mesh or foraminous membrane), polymer, or the like.

Alternatively, the filter could be self-expanding, e.g., formed from a shape memory alloy such as a nickel-titanium alloy (e.g., a Nitinol® or NiTi mesh), from a fabric or other sheet-like material with laser-formed or other holes (100 μm to 200 μm), usually having a nickel-titanium alloy or other elastic frame for deployment.

In in some instances, the filter basket could have a closure mechanism to close its opening before retraction, e.g., a cinch or “lasso” that can be used to close the opening with the emboli trapped in the filter.

In preferred instances, the filter will be delivered over a guidewire or balloon catheter through the center of a delivery system, e.g., a prosthetic valve delivery system.

In other instances, the filter will be constructed and delivered separately from any valve delivery or valve repair system.

The filters will be delivered to the atria through the venous vasculature, typically through a vena cava, usually the inferior vena cava. For example, a guidewire may be advanced through the left or right atrium into the to the left or right ventricle to cover an opening of, or be further advanced into a root of, the Aorta or Pulmonary artery The guidewire may be same guidewire used for advancing a repair tool or new valve delivery system. In some instances, the filter may be folded or otherwise collapsed and placed in or through a lumen of a delivery or repair system.

After crossing the atrial septum in the case of mitral valve replacement, the filter may be advanced to sit in the root of the aorta (distal to the aortic valve) and or may be deployed over an opening of the aortic valve in the left ventricular wall. When deployed over the valve opening, the filter basket may further extend over a portion of, or in some cases, over most or all of, the left ventricular wall except for the outlet of the tricuspid valve.

In some instances, the filter may be delivered in a separate delivery catheter and, when in the right location, released from constraint (e.g., unsheathed) and allowed to self-expand to form the basket shape.

Alternatively, the filter may be folded over a deflated balloon, and the balloon may be advanced to a position over or into the root of the target aortic or pulmonary valve. The balloon may be inflated to expand and/or shape the basket filter. The balloon will be deflated and removed, leaving the basket in place. The basket is typically be connected to one or more wires or tethers to allow retraction at the end of the procedure. Optionally, the wires or tethers may be used as “guidewires” to advance interventional tools for repairing or replacing the atrial outlet valve.

In one option there will be a lasso or other closure mechanism to close the opening of the basket at the end of the procedure to prevent loss of particles that had been captured in the basket.

In other instances, the filter will be inserted first over a guidewire in a separate delivery sheath (together with a balloon in case of balloon-expanded filters), and the sheath may be retracted, leaving the filter and retraction wire together with the guidewire in place. The interventional tools, for example a valve delivery system or a leaflet closure device, such as the MitraClip® system (available from Abbott) will be inserted over the retraction wires or tethers.

In a first aspect, the present invention provides a method for treating an atrial heart valve between a patient's atrium and ventricle. The method comprises placing a filter in or over a ventricular heart valve, i.e., a heart valve which establishes blood flow from the patient's ventricle into the patient's vasculature, such as the aortic valve or the pulmonary valve. The atrial heart valve is treated while the filter remains over the ventricular heart valve, such that emboli released into the ventricle while treating the atrial heart valve is captured by the filter during ventricular systole when blood flows from the ventricle through the filter into the vasculature. The filter together with the captured emboli is removed after the treatment is complete.

In some instances, the filter is placed over a ventricular side of the ventricular heart valve. Optionally, the filter may further cover a portion of a wall of the ventricle surrounding the ventricular heart valve. The filter may be configured to anchor on the ventricular wall so that the filter will remain in place while attached only to the retention wires or tethers.

In some instances, the filter is placed into a root of the ventricular heart valve.

In some instances, the atrial heart valve is a mitral valve and the ventricular heart valve is an aortic valve.

In some instances, the atrial heart valve is a tricuspid valve, and the ventricular heart valve is a pulmonary valve.

In some instances, the filter is secured prior to filter removal in order to prevent release of emboli as the filter is removed.

In some instances, the filter comprises a foraminous basket having an open end oriented to receive blood flaw and emboli. In such instances, securing the filter to prevent release of emboli may comprise cinching a loop surrounding the open end.

In some instances, placing the filter comprises advancing the filter through a vena cava into a right atrium. In such instances, the filter may be further advanced through a tricuspid valve into the right ventricle and then placed over or into a pulmonary valve, and a tricuspid valve implantation or repair procedure may be performed.

In some instances, the filter is may be further advanced transeptally into a left atrium and through a mitral valve into the left ventricle and then placed over or into an aortic valve. In such instances a mitral valve implantation or repair procedure may be performed.

In a second aspect of the present invention, a filter assembly comprises an elongate support element and a filter assembly. The elongate support element has a distal end and is configured to be advanced through a patient's vena cava through the patient's heart atrium into the patient's heart ventricle. The filter is attached to the distal end of the elongate support element and comprises a foraminous basket having an open end. The foraminous basket is configured to be placed in or over a ventricular heart valve which establishes blood flow from the heart ventricle into the patient's vasculature. In this way, emboli released into the ventricle while treating an atrial heart valve is captured by the filter during ventricular systole when blood flows from the ventricle through the filter into the patient's vasculature.

In some instances, the filter may be configured to be placed over a ventricular side of the ventricular heart valve, and optionally the filter may further be configured to cover a portion of a wall of the ventricle surrounding the ventricular heart valve.

In other instances, the filter may be configured to be placed into a root of the ventricular heart valve. When the filter is deployed in the ventricle, the filter provides protection to the coronary vessels that are not protected when the device is placed in the aorta since the of the coronary artery opening are located on the aortic artery side of the leaflets of the aortic valve.

In preferred instances, the open end of the filter is configured to be closed to secure emboli therein prior to removal from the patient. For example, the filter may further comprise a cinching loop circumscribing the open end of the filter.

### BRIEF DESCRIPTION OF THE DRAWINGS

FIG. 1 illustrates the anatomy of a human heart showing the locations of the heart chambers and heart valves.

FIG. 2 illustrates an exemplary filter assembly constructed in accordance with the principles of the present invention.

FIG. 3 illustrates a system for delivering the filter assembly of FIG. 2.

FIGS. 4A to 4D illustrate balloon expansion of the filter assembly of FIGS. 2 and 3.

FIGS. 5A to 5E illustrate use of the system of FIG. 3 for delivering the filter assembly of FIG. 2 to an aortic valve to capture emboli released in a mitral valve procedure.

FIGS. 6A to 6C illustrate use of the system of FIG. 3 for delivering the filter assembly of FIG. 2 to a pulmonary valve to capture emboli released in a tricuspid valve procedure.

### DETAILED DESCRIPTION OF THE INVENTION

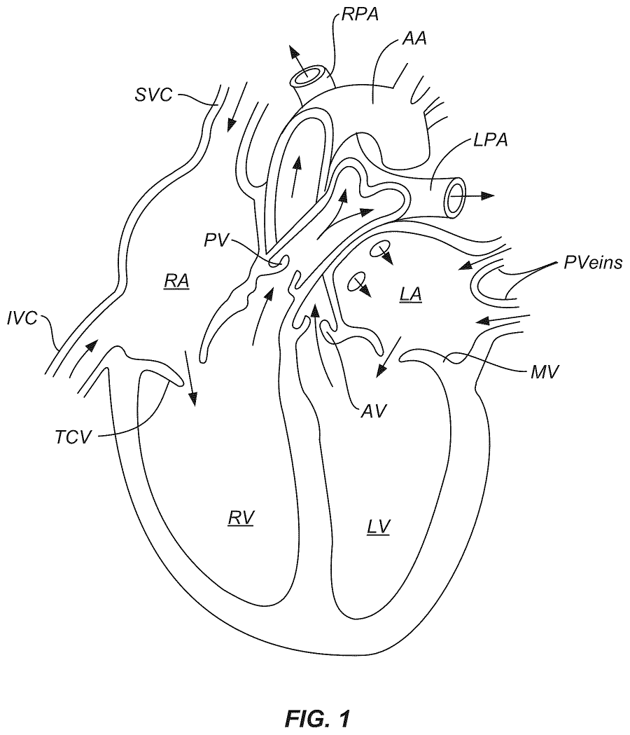

Referring now to FIG. 1, the human heart includes four chambers: the right atrium RA, the right ventricle RV, the left atrium LA, and the left ventricle LV. Venous blood enters the right atrium RA via the inferior vena cava IVC and the superior vena cava SVC and flows into the right ventricle RV through the tricuspid valve TCV during atrial systole. From the right ventricle RV, the venous blood flows to the lungs through the pulmonary valve PV and the pulmonary arteries RPA and LPA. After being oxygenated in the lungs, the blood enters the left atrium LA through four pulmonary veins PV. From the left atrium, the oxygenated blood flows into the left ventricle LV, through the mitral valve MV during atrial systole. The oxygenated blood then flows from the left ventricle LV into the aortic arch AA through the aortic valve AV and then into patient circulation.

A variety of procedures may be performed to replace or repair defective mitral valves MV and tricuspid valves TCV. For example, mitral valves MV maybe replaced with prosthetic valves during closed chest, beating heart procedures using catheters in a variety of ways. Such procedures, however, risk releasing emboli into the left ventricle LV from where it can pass through the aortic valve AV into the aortic arch AA and then to patient circulation. This is a particular risk to the cerebral vasculature where the emboli may flow through the side branch vessels on the aortic arch AA. Similarly, when procedures are performed on the tricuspid valve TCV, emboli may be released into the right ventricle RV, pass into the patient's lungs through the pulmonary valve LV and pulmonary arteries RPA and LPA, placing the patient at risk of pulmonary embolism.

The purpose of the present invention is to place a filter element at locations in and around the aortic valve AV and, in other cases, the pulmonary valve PV to prevent intrusion of emboli from the left and right ventricles, respectively, during ventricular systole.

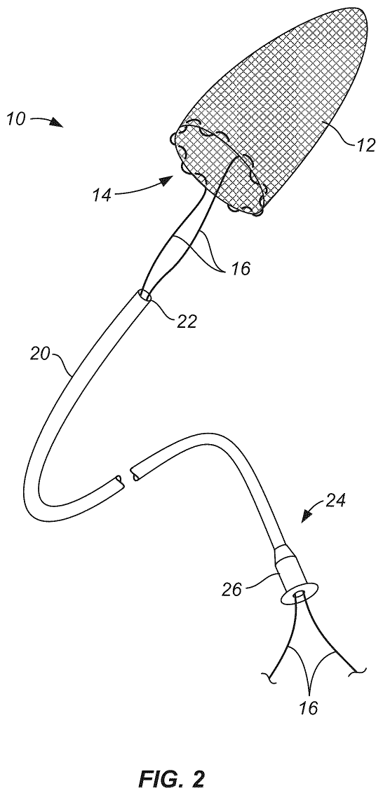

A suitable filter assembly 10 for use in the methods of the present invention is illustrated in FIG. 2. The filter assembly 10 comprises a filter basket 12 having an open end 14 which is configured to allow blood flow to enter an interior of the filter basket where it can be captured by the porous filter medium. The filter basket 12 is retained and controlled buy a pair of retention wires or tethers 16, each of which extends around a portion of the perimeter of the open end 14 as illustrated. Typically, the retention wire 16 will be connected in a “purse string” manner which allows them to be cinched to close the open end 14 after emboli have been captured in the interior of the filter basket 12. The filter basket 12 is illustrated as and elongate tapered body with a closed distal end, but other configurations could be used as well.

Often, the open end 14 will include a frame or other support (not illustrated) that holds the end 14 open and, optionally, may include barbs, hooks, screws, or other anchors (not shown) which are configured to hold the filter basket 14 in place after initial deployment. In this way, the filter basket 14 can be deployed and the delivery system party or wholly retracted, leaving the filter basket connected only by the wires or tethers 16. The wires or tethers 16 may also be configured to act as guidewires for the advancement of separate interventional or repair tools.

The filter basket 12 is deployed using a filter delivery sheath 20 having an open distal end 22 and a proximal end 24, a hub 26 is mounted on the proximal end 24 of the delivery sheath 20, and includes a single port allowing passage of the retention wire 16 as well as guidewires, valve interventional catheters, and the like.

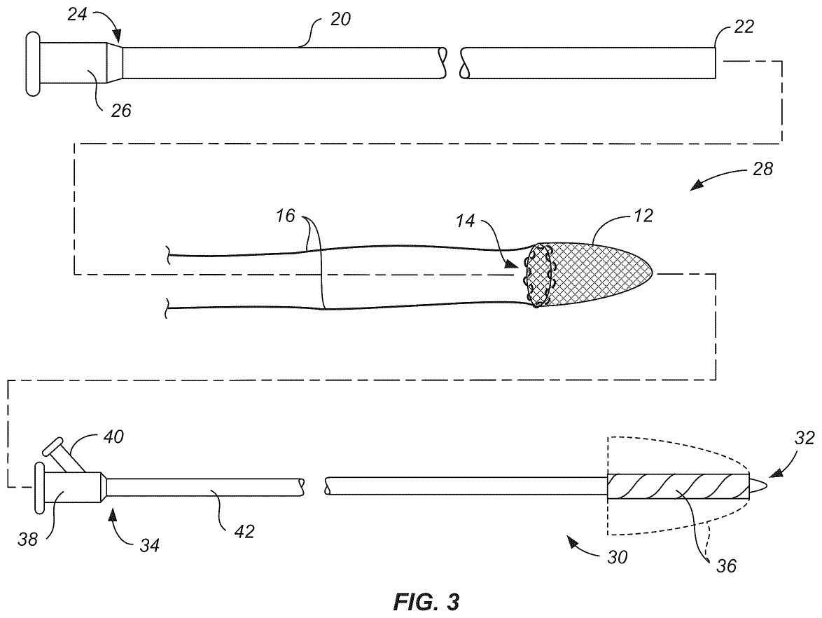

The filter assembly 10 may be provided as part of a delivery system 28 as illustrated in FIG. 3. In addition to the filter assembly 10, the delivery system 28 includes a balloon catheter 30 comprising a shaft 42 having a distal end 32 and a proximal end 34. A balloon 36 is located at the distal end 32 of the catheter shaft 42 and is configured to be inflated from a non-inflated condition, as illustrated in full line, to an elongated tapered shape similar or identical to the intended shape of the filter basket 12, as illustrated in broken line. The bloom catheter 30 further includes a proximal hub 38 having a side port 40 for inflating the balloon.

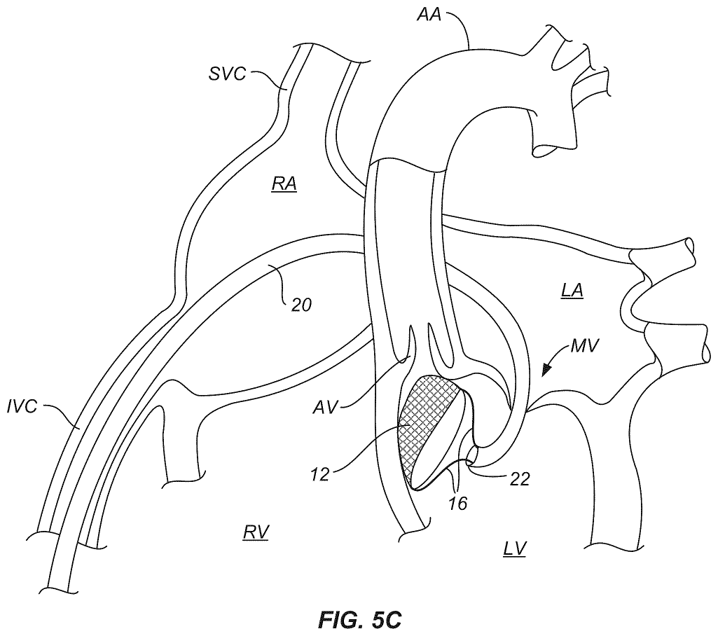

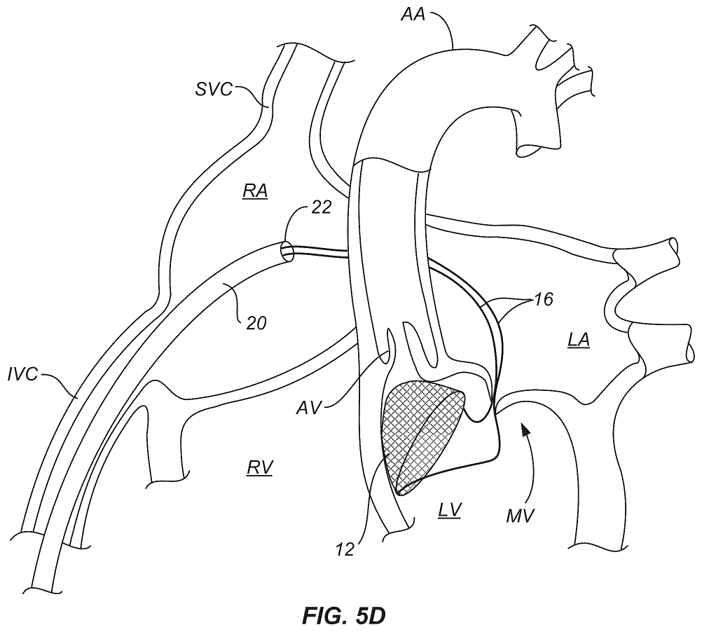

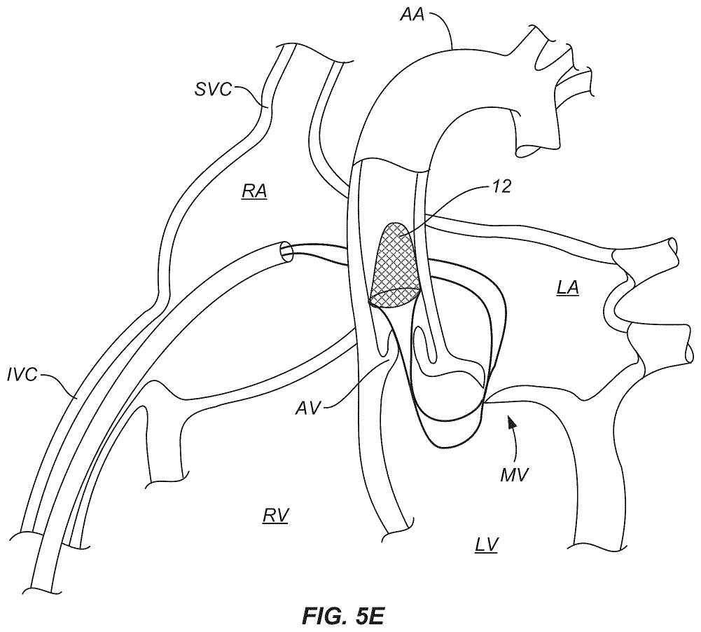

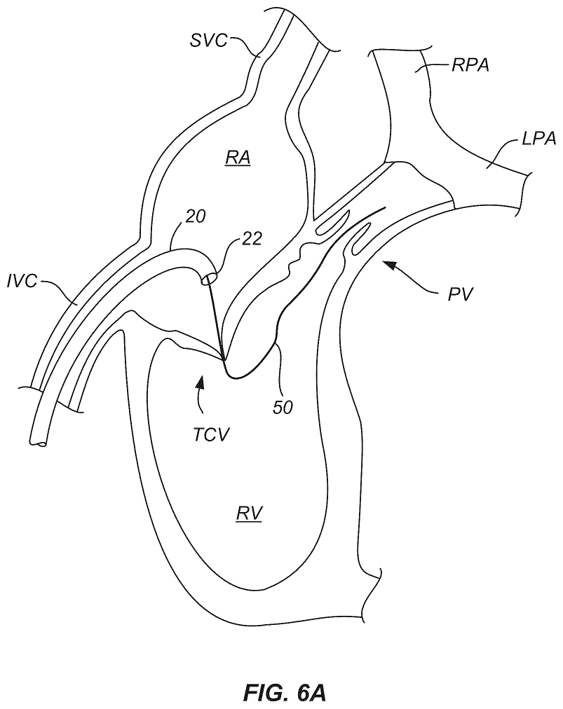

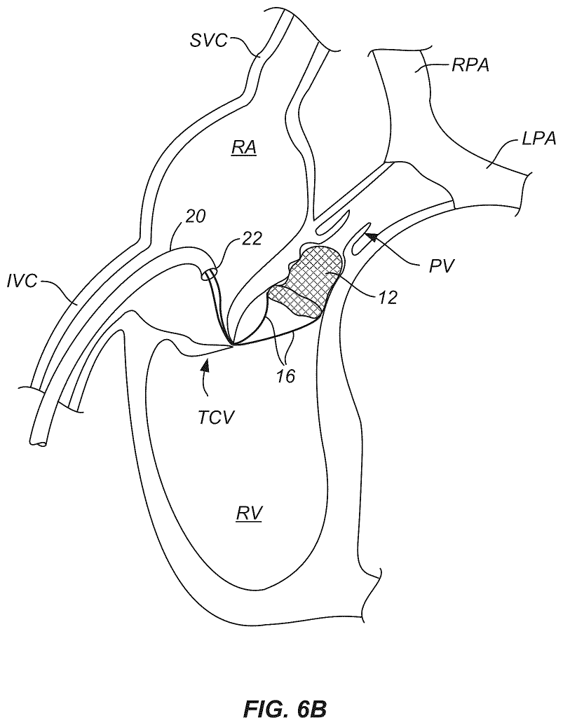

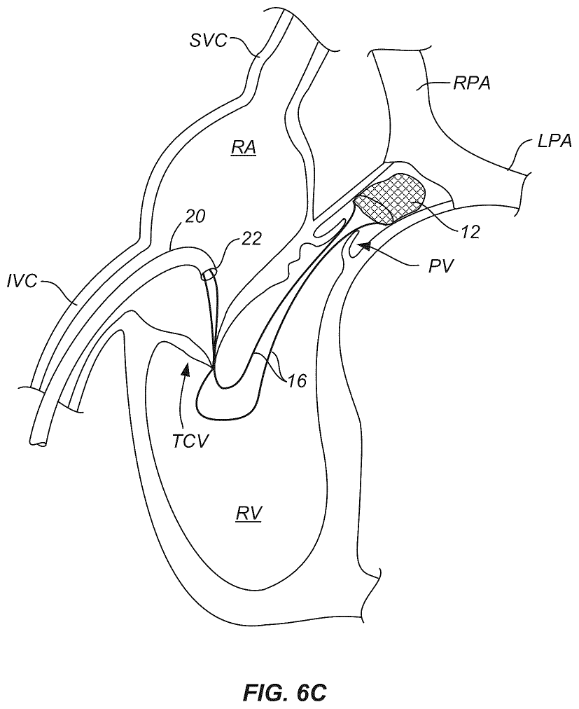

In some instances, the filter delivery sheath 20 may be sized, shaped, or otherwise configured to allow for advancement of an interventional or repair tool thereover. For example, as shown in FIGS. 5D, 5E, 6B, and 6C, the delivery sheath 20 may be retracted back into the right or left atrium RA or LA, leaving the distal end 22 of the delivery sheath 20 in the atrium and the filter basket 12 deployed over or in the ventricular outlet valve and connected to the deployment sheath only by wire/tethers 16. When in such a configuration, a separate interventional or repair tool (not shown) may be advanced over the sheath 20 and then over a portion of the wires 16 to reach the target atrial outlet valve.

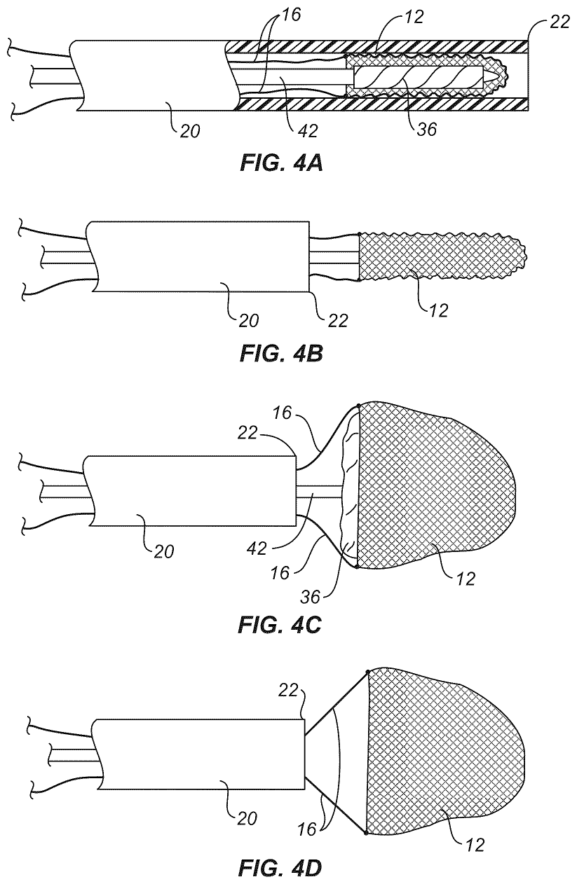

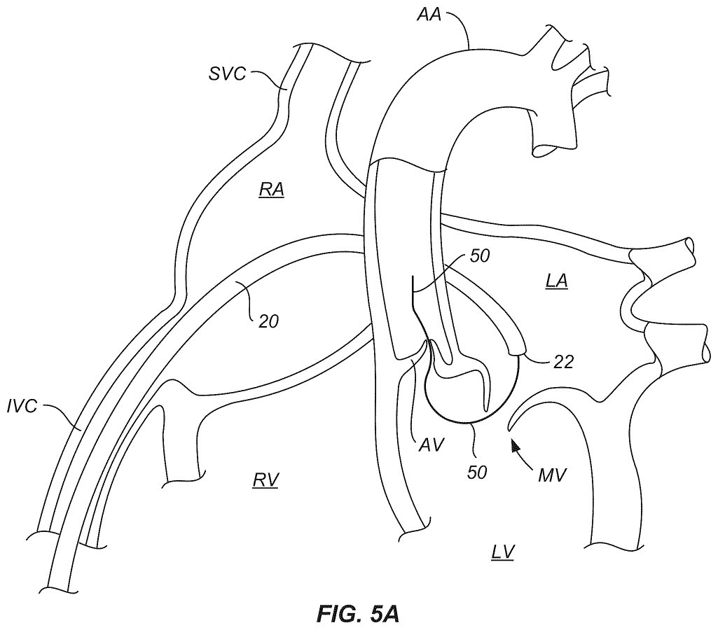

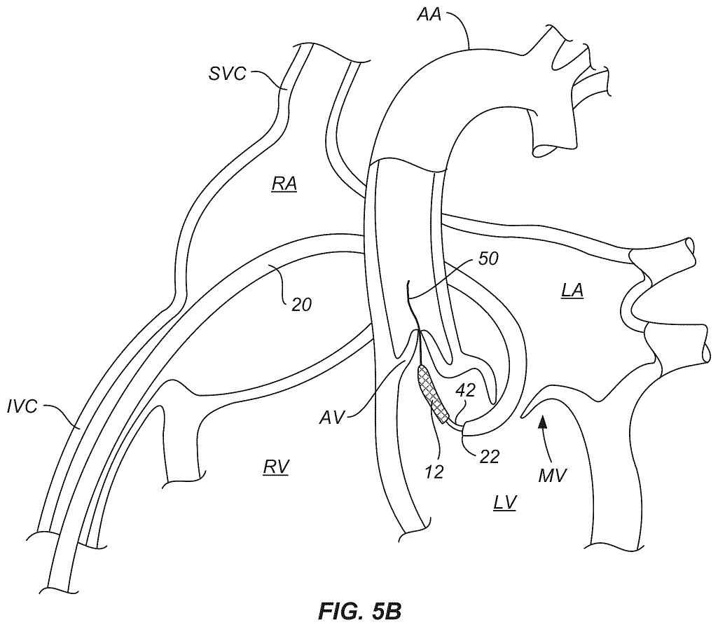

Referring now to FIGS. 4A to 4D, in preferred examples, the filter basket 12 is mounted over the balloon 36 and held within the distal end 22 of the filter delivery sheath 20. The filter basket 12 may be deployed by first pushing distally on balloon catheter shaft 42 so that the filter basket 12 emerges from the distal end 22 of the sheath, as shown in FIG. 4B. Balloon 36 may then be inflated, as shown in FIG. 4C, so that the filter basket 12 achieves the desired configuration. After the filter basket 12 has been properly deployed, the balloon 36 may be withdrawn from the sheath 20 by removing the entire balloon catheter 30. The filter basket 12 is then retained solely by the retention wires/tethers 16 which pass through the delivery sheath 20 and are available not only to manipulate the filter basket 12 but also to, optionally, introduce interventional tools thereover.

While use of a balloon is often preferred for deploying the filter basket 12, in other instances as described above, the filter basket may be self-expanding and deployed simply by being pushed from the distal end 22 of the delivery sheath 20.

Referring now FIGS. 5A to 5E, the filter basket 12 may be delivered to an aortic valve AV during performance of a procedure on a mitral valve MV by advancing the filter delivery sheath 20 through the inferior vena cava IV and into the right atrium RA using conventional intravascular delivery techniques. After entering the right atrium RA, the distal end 22 of the filter delivery sheath 20 is advanced across the atrial septum (not shown) and into the left atrium LA, as shown in FIG. 5A. The advancement will typically be done over a guidewire 50.

As shown in FIG. 5B, the filter basket 12 in its non-deployed configuration is advanced over the guidewire 50 to a position just beneath the leaflets of the aortic valve AV. The balloon 36 may then be inflated to deploy the filter basket 12, and the balloon catheter 30 may then be removed, as shown in FIG. 5C. Prior to performing a procedure on the mitral valve MV, to avoid interference, it is frequently desirable to retract the distal end 22 of the delivery sheath 20 back into the right atrium RA, as shown in FIG. 5D.

When deployed as shown in FIG. 5D, the filter basket 12 covers the ventricular side of the aortic valve AV, typically extending over an adjacent annular margin of the ventricular wall.

This positioning is advantageous has it prevents or inhibits emboli from entering any portion of the aortic root and aortic arch AA. In some instances, however, it may be preferable to have deployed the filter basket 12 within the aortic root, as shown in FIG. 5E. Such positioning will provide protection against emboli release into the aortic arch and, more importantly, into the side branches of the aortic arch. Such positioning in t aortic root, however, will not fully protect the coronary arteries which will typically be located below the deployed region of the filter basket 12.

Referring now to FIGS. 6A to 6C, the filter basket 12 may be delivered to a pulmonary valve PV during performance of a procedure on a tricuspid valve TCV by advancing the filter delivery sheath 20 through the inferior vena cava IVC and into the right atrium RA so that the distal end 22 of the sheath lies over the tricuspid valve, as shown in FIG. 6A. The balloon catheter 30 (not shown) may then be advanced over the guidewire 50 to deploy the filter basket 12 to cover the ventricular side of the pulmonary valve PV, as shown in FIG. 6B. Alternatively, the balloon catheter 30 may be further advanced through the pulmonary valve PV in order to deploy the filter basket 12 within the root of the pulmonary artery, as shown in FIG. 6C.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for treating an atrial heart valve between a patient's atrium and ventricle, said method comprising:
placing a filter in or over a ventricular heart valve which establishes blood flow from the patient's ventricle into the patient's vasculature;
treating the atrial heart valve while the filter remains over the ventricular heart valve, wherein emboli released into the ventricle while treating the atrial heart valve is captured by the filter during ventricular systole when blood flows from the ventricle through the filter into the vasculature; and
removing the filter together with the captured emboli.

2. The method of claim 1, wherein the filter is placed over a ventricular side of the ventricular heart valve.

3. The method of claim 2, wherein the filter further covers a portion of a wall of the ventricle surrounding the ventricular heart valve.

4. The method of claim 1, wherein the filter is placed into a root of the ventricular heart valve.

5. The method of claim 1, wherein the atrial heart valve is a mitral valve and the ventricular heart valve is an aortic valve.

6. The method of claim 1, wherein the atrial heart valve is a tricuspid valve and the ventricular heart valve is a pulmonary valve.

7. The method of claim 1, further comprising securing the filter to prevent release of emboli before removing the filter.

8. The method of claim 7, wherein the filter comprises a foraminous basket having an open end oriented to receive blood flaw and emboli

9. The method of claim 8, wherein securing the filter to prevent release of emboli comprises cinching a loop surrounding the open end.

10. The method of claim 1, wherein placing the filter comprises advancing the filter through a vena cava into a right atrium.

11. The method of claim 10, wherein the filter is further advanced through a tricuspid valve into the right ventricle and then placed over or into a pulmonary valve.

12. The method of claim 11, further comprising performing a tricuspid valve implantation or repair procedure.

13. The method of claim 10, wherein the filter is further advanced transeptally into a left atrium and through a mitral valve into the left ventricle and then placed over or into an aortic valve.

14. The method of claim 13, further comprising performing a mitral valve implantation or repair procedure.

15. A filter assembly comprising:
an elongate support element having a distal end configured to be advanced through a patient's vena cava through the patient's heart atrium into the patient's heart ventricle;
a filter comprising a foraminous basket having an open end secured to the distal end of the elongate support element;
wherein the foraminous basket is configured to be placed in or over a ventricular heart valve which establishes blood flow from the heart ventricle into the patient's vasculature;
wherein emboli released into the ventricle while treating an atrial heart valve is captured by the filter during ventricular systole when blood flows from the ventricle through the filter into the patient's vasculature.

16. The filter assembly of claim 15, wherein the filter is configured to be placed over a ventricular side of the ventricular heart valve.

17. The filter assembly of claim 16, wherein the filter is further configured to cover a portion of a wall of the ventricle surrounding the ventricular heart valve.

18. The filter assembly of claim 16, wherein the filter is configured to be placed into a root of the ventricular heart valve.

19. The filter assembly of claim 15, wherein the open end is configured to be closed to secure emboli therein prior to removal from the patient.

20. The filter assembly of claim 19, wherein the filter further comprises a cinching loop circumscribing the open end of the filter.
